(19) United States
(12) Patent Application Publication
(10) Pub. No.: US 2012/0195876 A1
(43) Pub. Date: Aug. 2, 2012

(54) NOVEL ROLE OF ALPHA-GALACTOSIDASE ACTIVITY AS A BIOMARKER IN KIDNEY DISEASE

(76) Inventor: Jochen Reiser, Miami, FL (US)

(21) Appl. No.: 13/390,272

(22) PCT Filed: Aug. 13, 2010
(86) PCT No.: PCT/US10/45413

§ 371 Date: Apr. 19, 2012

Related U.S. Application Data
(60) Provisional application No. 61/233,885, filed on Aug. 14, 2009.

(51) Int. Cl. A61K 38/47; A61P 13/12; A61P 35/00; C12N 9/40; C12Q 1/34

(52) U.S. Cl. 424/94.61; 435/208; 435/18

(57) ABSTRACT

Biomarkers for diagnosis and differentiation between stages of kidney diseases. Methods of treatments and identification of novel therapies are provided.

### CROSS REFERENCE TO RELATED APPLICATIONS

This application claims priority to U.S. provisional application No. 61/233,885, filed Aug. 14, 2009, which is incorporated herein by reference in its entirety.

### FIELD OF THE INVENTION

Embodiments of the invention are directed to biomarkers of early chronic kidney diseases. Methods of identifying at risk patients and the early diagnosis of kidney disease are provided.

### BACKGROUND

Lysosomal enzyme alpha-galactosidase A (alpha-GAL) is the enzyme that is mutated in Fabry disease, also known as angiokeratoma corporis diffusum universale. Fabry disease was first described independently by Drs. William Anderson in England and Johann Fabry in Germany in 1898. Fabry disease is a rare, panethnic, X-linked recessive lysosomal storage disorder caused by a deficiency of the lysosomal enzyme alpha-galactosidase A (alpha-GAL). Partial or complete deficiency of alpha-GAL leads to progressive accumulation of glycosphingolipids, particularly globotriaosylceramide (GL-3), in visceral tissues, the kidney and the vascular endothelium throughout the body and can be associated with progressive kidney failure. Fabry disease is a rare hereditary disorder. It is estimated that 1 in 40,000 males has Fabry disease, whereas the estimated prevalence in the general population is 1 in 117,000 people.

### SUMMARY

This Summary is provided to present a summary of the invention to briefly indicate the nature and substance of the invention. It is submitted with the understanding that it will not be used to interpret or limit the scope or meaning of the claims.

Compositions for the diagnosis of kidney diseases and disorders comprise at least one biomarker which is correlated with the disease.

In a preferred embodiment, a biomarker comprising alpha-galactosidase expression and/or activity is diagnostic of early stage chronic kidney disease.

In another preferred embodiment, a biomarker comprising alpha-galactosidase expression and/or activity is diagnostic of acute renal failure and/or indicative of an individual at risk of developing acute renal failure.

Methods of treatment comprise administration of alpha-galactosidase and/or agents which modulate expression and/or activity of alpha-galactosidase.

High-throughput screening assays identify novel therapeutic agents.

Other aspects are described infra.

### BRIEF DESCRIPTION OF THE DRAWINGS

FIG. 1 shows the assay scores for 300 normal controls from Genzyme (left), 50 samples from ProMedDx vacutainer flasks (middle) and 20 finger tip samples collected at the Massachusetts General Hospital (right). The difference in median values for the latter two groups (45 vs 21 pmol/punch/h) are due to the fact that amount of blood dried per unit area (preprinted circle) on the filter paper is higher (70 μl) for samples from vacutainer than those (30-40 μl) from finger tips. Even if the blood spot disc was punched out from an area completely impregnated with blood, the amount of blood per ‘circle’ is still important which causes variations in the enzyme activity levels between two methods of specimen preparation. Since the clinical data was collected by ‘finger tip’ method, the normal control data for ‘finger tip’ will be used while screening the populations in order to implement a built-in corrective mechanism.

FIG. 2 shows assay scores for 300 normal controls compared to 18 male Fabry patients collected by Genzyme (left) and 20 normal controls compared to 4 male Fabry patients collected at the Massachusetts General Hospital (right). The cutoff values are 13 and 11 pmol/punch/h, respectively.

FIG. 3 shows assay scores for 300 normal controls compared to 55 Fabry disease carriers collected by Genzyme (left) and 20 normal controls compared to 7 Fabry disease carriers collected at the Massachusetts General Hospital (right).

FIG. 4 is a graph showing alpha-galactosidase levels at different stages of CKD.

### DETAILED DESCRIPTION

Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. The present invention is not limited by the illustrated ordering of acts or events, as some acts may occur in different orders and/or concurrently with other acts or events. Furthermore, not all illustrated acts or events are required to implement a methodology in accordance with the present invention.

All genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, for the genes disclosed herein, which in some embodiments relate to mammalian nucleic acid and amino acid sequences are intended to encompass homologous and/or orthologous genes and gene products from other animals including, but not limited to other mammals, fish, amphibians, reptiles, and birds. In preferred embodiments, the genes or nucleic acid sequences are human.

### DEFINITIONS

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, preferably up to 5%, or preferably up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

Expression/amount of a gene, biomolecule, or biomarker in a first sample is at a level “greater than” the level in a second sample if the expression level/amount of the gene or biomarker in the first sample is at least about 1 time, 1.2 times, 1.5 times, 1.75 times, 2 times, 3 times, 4 times, 5 times, 6 times, 7 times, 8 times, 9 times, 10 times, 20 times, 30 times, the expression level/amount of the gene or biomarker in the second sample or a normal sample. Expression levels/amounts can be determined based on any suitable criterion known in the art, including but not limited to mRNA, cDNA, proteins, protein fragments and/or gene copy. Expression levels/amounts can be determined qualitatively and/or quantitatively.

By the term “modulate,” it is meant that any of the activities, are, e.g., increased, enhanced, increased, agonized (acts as an agonist), promoted, decreased, reduced, suppressed blocked, or antagonized (acts as an agonist). Modulation can increase activity more than 1-fold, 2-fold, 3-fold, 5-fold, 10-fold, 100-fold, etc., over baseline values. Modulation can also decrease its activity below baseline values.

An “allele” or “variant” is an alternative form of a gene. Variants may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to variants are generally ascribed to natural deletions, additions, or substitutions of nucleotides. Each of these types of changes may occur alone, or in combination with the others, one or more times in a given sequence.

“Biological samples” include solid and body fluid samples. Preferably, the sample is obtained from the kidney. However, the biological samples used in the present invention can include cells, protein or membrane extracts of cells, blood or biological fluids such as ascites fluid or brain fluid (e.g., cerebrospinal fluid). Examples of solid biological samples include, but are not limited to, samples taken from tissues of the central nervous system, bone, breast, kidney, cervix, endometrium, head/neck, gallbladder, parotid gland, prostate, pituitary gland, muscle, esophagus, stomach, small intestine, colon, liver, spleen, pancreas, thyroid, heart, lung, bladder, adipose, lymph node, uterus, ovary, adrenal gland, testes, tonsils and thymus. Examples of “body fluid samples” include, but are not limited to blood, serum, semen, prostate fluid, seminal fluid, urine, saliva, sputum, mucus, bone marrow, lymph, and tears.

“Sample” is used herein in its broadest sense. A sample comprising polynucleotides, polypeptides, peptides, antibodies and the like may comprise a bodily fluid; a soluble fraction of a cell preparation, or media in which cells were grown; a chromosome, an organelle, or membrane isolated or extracted from a cell; genomic DNA, RNA, or cDNA, polypeptides, or peptides in solution or bound to a substrate; a cell; a tissue; a tissue print; a fingerprint, skin or hair; and the like.

“Diagnostic” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

“Treating” or “treatment” covers the treatment of a disease-state in a mammal, and includes: (a) preventing the disease-state from occurring in a mammal, in particular, when such mammal is predisposed to the disease-state but has not yet been diagnosed as having it; (b) inhibiting the disease-state, e.g., arresting it development; and/or (c) relieving the disease-state, e.g., causing regression of the disease state until a desired endpoint is reached. Treating also includes the amelioration of a symptom of a disease (e.g., lessen the pain or discomfort), wherein such amelioration may or may not be directly affecting the disease (e.g., cause, transmission, expression, etc.).

As used herein, the term “individual”, “animal” or “patient” is meant to include, for example, humans, sheep, elks, deer, mule deer, minks, mammals, monkeys, horses, cattle, pigs, goats, dogs, cats, rats, mice, birds, chicken, reptiles, fish, insects and arachnids. “Mammal” covers warm blooded mammals that are typically under medical care (e.g., humans and domesticated animals). Examples include feline, canine, equine, bovine, and human, as well as just human. Preferably, the patient is human.

### Biomarkers

Fabry disease is a rare, pan ethnic, X-linked recessive lysosomal storage disorder caused by a deficiency of the lysosomal enzyme alpha-galactosidase A (alpha-GAL). Partial or complete deficiency of alpha-GAL leads to progressive accumulation of glycosphingolipids, particularly globotriaosylceramide (GL-3), in visceral tissues and the vascular endothelium throughout the body.

The inability to catabolize GL-3 leads to progressive multisystemic damage to the kidney, heart, and cerebrovascular system. The clinical course of Fabry disease is usually marked by chronic pain, angiokeratomas, hypohidrosis, heat and cold intolerance, corneal opacities, renal failure, stroke, and cardiac complications. As the disease progresses, complications may become life-threatening.

Since Fabry is X-linked, the disease predominantly affects males (hemizygotes), who have little if any endogenous alpha-GAL. Although X-linked recessive diseases generally do not affect females, there are female carriers (heterozygotes) who may experience varying degrees of disease manifestations. It is believed that X-chromosomal inactivation (lyonization), which can block expression of the functional alpha-GAL gene in all or some parts of the body, is responsible for disease onset in carriers. Although the prevalence of female carriers who develop overt clinical manifestations is unknown, recent studies indicate that manifestations in carrier females are more common than previously thought.

Clinical presentation and sequelae: Although classical Fabry disease usually presents in childhood (with pain, fever, hypohidrosis, fatigue, and exercise intolerance) diagnosis may not be confirmed until there has been considerable accumulation of GL-3. The average age of diagnosis is approximately 30 years. Delayed diagnosis may be due in part to under-recognition of the disease and/or misattribution of Fabry disease symptoms to other disorders.

Progressive organ and tissue damage associated with Fabry disease may result in substantially decreased life expectancy. Before the availability of renal dialysis or transplantation, the average age of death among patients with classical Fabry disease was 41 years; today, average life expectancy is still only 50 years.

Atypical Variant: Growing evidence indicates there may be a significant number of “atypical variants”—hemizygotes who have few or none of the hallmark symptoms of classical Fabry disease. Atypical variants have residual plasma alpha-GAL levels (1% to 30% of normal) and present much later in life than patients with classical Fabry disease. They are often identified serendipitously, and usually have manifestations predominately in one organ system.

The disease is rare with an incidence of 0.2-0.8 per 100,000 newborns. Affected individuals can be found among patients with renal, cardiac or cerebrovascular disease. Since no single predominant mutation is found in Fabry disease, screening for mutations is far too laborious. The availability of a treatment with alpha-GAL infusions has greatly increased the need to identify patients with this disorder.

In a preferred embodiment, a biomarker comprising alpha-galactosidase is diagnostic of chronic kidney disease. The studies herein address the lack of biomarkers that can with great sensitivity and specificity distinguish healthy people from patients with chronic kidney disease (CKD) stages 1 or 2 (CKD 1 or 2). Especially, the differentiation in these groups is so important because an early intervention can save kidney function. The routine scenario is that a patient is diagnosed with kidney disease at stage 3 or 4 and at this point, therapeutic options are already limited.

Briefly, the data show that alpha-galactosidase enzymatic activity in leukocytes is extremely sensitive to changes in kidney function and differentiates in experiments well between normal patients or patients with CKD stage 1 (normal renal function) and CKD stage 2. Initially, a simple, cheap and reliable test was sought to potentially identify new Fabry-patients since many patients that present with some generalized symptoms (e.g. proteinuria) could also be considered potential carriers of the defective alpha-galactosidase enzyme. A dried blood spot test system was developed where blood from patients (finger prick or venous blood) was transferred on a Whatman paper. The dried blood spot can be stored at room temperature and at the convenience of the user, lysed with a buffer. Then, X-gal is given to the reaction as a substrate and the leukocyte alpha-galactosidase enzymatic activity is determined. The initial studies were extended to analyzing enzymatic activity levels of alpha-galactosidase in patients with various stages of Chronic Kidney Disease (CKD). Usually, CKD is categorized in 5 stages (see Table 1).

TABLE 1
STAGES OF CHRONIC KIDNEY DISEASE (CDK)

| Stage | Description | GFR Level | |
|---|---|---|---|
| Normal | Healthy kidneys | 90 | mL/min or more |
| kidney | | | |
| function | | | |
| Stage 1 | Kidney damage with | 90 | mL/min or more |
| | normal or high GFR | | |
| Stage 2 | Kidney damage and mild | 60 to 89 | mL/min |
| | decrease in GFR | | |
| Stage 3 | Moderate decrease in GFR | 30 to 59 | mL/min |
| Stage 4 | Severe decrease in GFR | 15 to 29 | mL/min |
| Stage 5 | Kidney failure | Less than 15 | mL/min |
| | | | or on dialysis |

In a preferred embodiment a biomarker for the diagnosis of early chronic kidney diseases comprises alpha-galactosidase, fragments, alleles, mutants, variants and fragments thereof. The detection, or lack thereof of alpha-galactosidase, can be expression of nucleic acids or peptides, enzyme activity, enzyme amounts, function and the like. Heretofore, no biomarkers were available that can with great sensitivity and specificity separate healthy people from patients with CKD 1 or 2. Especially, the differentiation in these groups is so important because an early intervention can safe kidney function. The routine scenario is that a patient is diagnosed with kidney disease at stage 3 or 4 and at this point, therapeutic options are already limited. The data in the examples section which follows, show that alpha-galactosidase enzymatic activity in leukocytes is extremely sensitive to changes in kidney function and differentiates in experiments well between normal patients or patients with CKD stage 1 (normal renal function) and CKD stage 2 (see examples section which follows).

In a preferred embodiment, a biomarker for the diagnosis of early kidney disease comprises: alpha-galactosidase, mutants, fragments, variants or derivatives thereof. The alpha-galactosidase can be detected as, for example, an increase in expression, amount, function, activity and the like.

In another preferred embodiment, the alpha-galactosidase activity is modulated in patients versus controls and also between the different stages of kidney disease. In one embodiment, as the kidney disease progresses through the various stages, the alpha-galactosidase activity decreases. Thus, in preferred embodiments, the amounts of alpha-galactosidase activity, is correlated with different stages of kidney diseases.

In another preferred embodiment, alpha-galactosidase activity differentiates between healthy individuals and individuals at stage one early chronic kidney disease.

In another preferred embodiment, a biomarker that is diagnostic of kidney diseases and disorders comprises one or more markers, such as for example, alpha-galactosidase. Alpha-galactosidase can be detected by, for example, expression, function and/or activity. Examples of kidney diseases or disorders comprise: acute kidney failure, acute nephritic syndrome, analgesic nephropathy, atheroembolic renal disease, chronic kidney failure, chronic nephritis, congenital nephrotic syndrome, end-stage renal disease, Goodpasture syndrome, interstitial nephritis, kidney cancer, kidney damage, kidney infection, kidney injury, kidney stones, Lupus nephritis, membranoproliferative GN I, membranoproliferative GN II, membranous nephropathy, minimal change disease, necrotizing glomerulonephritis, nephroblastoma, nephrocalcinosis, nephrogenic diabetes insipidus, nephropathy, nephrosis (nephrotic syndrome), polycystic kidney disease, post-streptococcal GN, reflux nephropathy, renal artery embolism, renal artery stenosis, renal disorders, renal papillary necrosis, renal tubular acidosis type I, renal tubular acidosis type II, renal underperfusion, renal vein thrombosis.

In another preferred embodiment, a biomarker comprising alpha-galactosidase, mutants, fragments, variants or derivatives thereof and/or alpha-galactosidase activity is diagnostic of acute renal failure or can identify patients at risk of developing acute renal failure.

In another preferred embodiment, a biomarker that is diagnostic of alpha-galactosidase associated diseases and disorders comprises one or more markers, such as for example, alpha-galactosidase expression, function and/or activity. Examples of alpha-galactosidase diseases or disorders comprise, for example, Fabry disease.

In another preferred embodiment, a method of identifying biomarkers for diagnosis of early stage chronic kidney disease comprising: obtaining samples from patients; measuring alpha-galactosidase activity at different stages of kidney disease; correlating the alpha-galactosidase activity with each stage of kidney disease; identify a biomarker expression, function and/or activity and correlated with the stage of kidney disease.

In another preferred embodiment, a method of identifying biomarkers for diagnosis of acute renal failure and/or identification of individuals at risk of acute renal failure comprising: obtaining samples from patients; measuring alpha-galactosidase activity, expression or function; correlating the alpha-galactosidase activity, expression or function, with each stage of kidney disease; identify biomarkers which are expressed or an activity of any molecule which is detected and correlated with the stage of kidney disease.

In another preferred embodiment, a method of diagnosing and differentiating between patients at different stages of kidney disease comprising: detecting alpha-galactosidase activity, expression or function and/or a second or plurality of biomarkers. Preferably, the alpha-galactosidase activity is modulated during each stage of kidney disease.

In another preferred embodiment, a biomarker for diagnosing diseases or disorders associated with alpha-galactosidase comprises alpha-galactosidase. For example, congenital kidney diseases, non-Fabry and Fabry disease.

In another preferred embodiment, the biomarker comprising alpha-galactosidase identifies patients who are at risk of developing a kidney disease or disorder. For example, those patients can be from families with genetically inherited kidney diseases, alcoholics and drug users and the like.

In another preferred embodiment, a method of treating a patient with a disease or disorder comprises administering to the patient a therapeutically effective amount of alpha galactosidase. The alpha galactosidase can be administered as a pharmaceutical composition, may be a vector expressing alpha-galactosidase, may be any agent that stimulates in vivo production of alpha-galactosidase, can be administered as a cell producing alpha-galactosidase etc.

In another preferred embodiment, a method of identifying agents for treatment or prevention of kidney diseases or disorders comprising: contacting a biomarker indicative of kidney disease with an agent; measuring activity, expression and/or function of the biomarker in the presence or absence of the agent; correlating the biomarker activity with a normal control.

In another preferred embodiment, a method of identifying agents for treatment or prevention of diseases or disorders associated with alpha-galactosidase expression, function and/or activity, comprising: contacting a sample with an agent; measuring activity, expression and/or function of alpha-galactosidase in the presence or absence of the agent; correlating the activity, expression and/or function of alpha-galactosidase with a normal control.

In another preferred embodiment, the alpha-galactosidase expression, function and/or is optionally further correlated with one or more biomarkers.

In a preferred embodiment, a sample comprises: a cell, tissue, fluid, peptide, polypeptide, oligonucleotide, polynucleotide, organic molecules, inorganic molecules or combinations thereof.

In another preferred embodiment, the detection in a cell or patient of alpha-galactosidase activity, expression and/or function, is diagnostic of kidney diseases and/or disorders thereof. Preferably, the alpha-galactosidase activity, expression and/or function, are modulated at levels by at least between 1%, 2%, 5%, 10% in a cell or patient as compared to levels in a normal cell or normal subject; more preferably, the alpha-galactosidase activity, expression and/or function, are modulated by about 50% in a cell or a patient as compared to levels in a normal cell or normal subject; more preferably, the alpha-galactosidase activity, expression and/or function, are modulated by about 75% in a cell or a patient as compared to levels in a normal cell or normal subject. The term “modulated” refers to an increase or decrease in level, concentration, amount etc, as compared to a normal cell or normal healthy subject.

In another preferred embodiment, a biomarker comprises polynucleotides, oligonucleotides, peptides, polypeptides, variants, mutants or fragments of alpha-galactosidase.

Detection of Nucleic Acids and Proteins as Markers: The methods and assays disclosed herein are directed to the examination of biomarkers in a mammalian tissue or cell sample, wherein the determination of that expression, activity of one or more such biomarkers is predictive of prognostic outcome or diagnostic of kidney diseases and disorders.

Preferred embodiments in the identification of biomolecules, analytical methods etc, are described in detail in the Examples which follow. Other methods for detection of other biomarkers comprise detection based on a microarray chip.

Microarrays: In general, using nucleic acid microarrays test and control mRNA samples from test and control tissue samples are reverse transcribed and labeled to generate cDNA probes. The probes are then hybridized to an array of nucleic acids immobilized on a solid support. The array is configured such that the sequence and position of each member of the array is known. For example, a selection of genes that have potential to be expressed in certain disease states may be arrayed on a solid support. Hybridization of a labeled probe with a particular array member indicates that the sample from which the probe was derived expresses that gene. Differential gene expression analysis of disease tissue can provide valuable information. Microarray technology utilizes nucleic acid hybridization techniques and computing technology to evaluate the mRNA expression profile of thousands of genes within a single experiment (see, e.g., WO 01/75166 published Oct. 11, 2001; (See, for example, U.S. Pat. No. 5,700,637, U.S. Pat. No. 5,445,934, and U.S. Pat. No. 5,807,522, Lockart, Nature Biotechnology, 14:1675-1680 (1996); Cheung, V. G. et al., Nature Genetics 21(Suppl):15-19 (1999) for a discussion of array fabrication). DNA microarrays are miniature arrays containing gene fragments that are either synthesized directly onto or spotted onto glass or other substrates. Thousands of genes are usually represented in a single array. A typical microarray experiment involves the following steps: 1) preparation of fluorescently labeled target from RNA isolated from the sample, 2) hybridization of the labeled target to the microarray, 3) washing, staining, and scanning of the array, 4) analysis of the scanned image and 5) generation of gene expression profiles. Currently two main types of DNA microarrays are being used: oligonucleotide (usually 25 to 70 mers) arrays and gene expression arrays containing PCR products prepared from cDNAs. In forming an array, oligonucleotides can be either prefabricated and spotted to the surface or directly synthesized on to the surface (in situ). The Affymetrix GENECHIP™ system is a commercially available microarray system which comprises arrays fabricated by direct synthesis of oligonucleotides on a glass surface.

Probe/Gene Arrays: Oligonucleotides, usually 25 mers, are directly synthesized onto a glass wafer by a combination of semiconductor-based photolithography and solid phase chemical synthesis technologies. Each array contains up to 400,000 different oligonucleotides and each oligonucleotide is present in millions of copies. Since oligonucleotide probes are synthesized in known locations on the array, the hybridization patterns and signal intensities can be interpreted in terms of gene identity and relative expression levels by the Affymetrix Microarray Suite software. Each gene is represented on the array by a series of different oligonucleotide probes. Each probe pair consists of a perfect match oligonucleotide and a mismatch oligonucleotide. The perfect match probe has a sequence exactly complimentary to the particular gene and thus measures the expression of the gene. The mismatch probe differs from the perfect match probe by a single base substitution at the center base position, disturbing the binding of the target gene transcript. This helps to determine the background and nonspecific hybridization that contributes to the signal measured for the perfect match oligonucleotide. The Microarray Suite software subtracts the hybridization intensities of the mismatch probes from those of the perfect match probes to determine the absolute or specific intensity value for each probe set. Probes are chosen based on current information from GenBank and other nucleotide repositories. The sequences are believed to recognize unique regions of the 3′ end of the gene. A GeneChip Hybridization Oven (“rotisserie” oven) is used to carry out the hybridization of up to 64 arrays at one time. The fluidics station performs washing and staining of the probe arrays. It is completely automated and contains four modules, with each module holding one probe array. Each module is controlled independently through Microarray Suite software using preprogrammed fluidics protocols. The scanner is a confocal laser fluorescence scanner which measures fluorescence intensity emitted by the labeled cRNA bound to the probe arrays. The computer workstation with Microarray Suite software controls the fluidics station and the scanner. Microarray Suite software can control up to eight fluidics stations using preprogrammed hybridization, wash, and stain protocols for the probe array. The software also acquires and converts hybridization intensity data into a presence/absence call for each gene using appropriate algorithms. Finally, the software detects changes in gene expression between experiments by comparison analysis and formats the output into .txt files, which can be used with other software programs for further data analysis.

The expression of a selected biomarker may also be assessed by examining gene deletion or gene amplification. Gene deletion or amplification may be measured by any one of a wide variety of protocols known in the art, for example, by conventional Southern blotting, Northern blotting to quantitate the transcription of mRNA (Thomas, Proc. Natl. Acad. Sci. USA, 77:5201-5205 (1980)), dot blotting (DNA analysis), or in situ hybridization (e.g., FISH), using an appropriately labeled probe, cytogenetic methods or comparative genomic hybridization (CGH) using an appropriately labeled probe.

Detection of Polypeptides: In another embodiment, a polypeptide corresponding to a marker is detected. A preferred agent for detecting a polypeptide of the invention is an antibody or aptamer capable of binding to a polypeptide corresponding to a marker of the invention, preferably an antibody with a detectable label. Antibodies can be polyclonal, or more preferably, monoclonal. An intact antibody, or a fragment thereof, e.g., Fab or F(ab′)₂ can be used. The term “labeled”, with regard to the probe or antibody, is intended to encompass direct-labeling of the probe or antibody by coupling, i.e., physically linking, a detectable substance to the probe or antibody, as well as indirect-labeling of the probe or antibody by reactivity with another reagent that is directly-labeled. Examples of indirect labeling include detection of a primary antibody using a fluorescently-labeled secondary antibody and end-labeling of a DNA probe with biotin such that it can be detected with fluorescently-labeled streptavidin.

Proteins from individuals can be isolated using techniques that are well-known to those of skill in the art. The protein isolation methods employed can, e.g., be such as those described in Harlow & Lane (1988), supra. A variety of formats can be employed to determine whether a sample contains a protein that binds to a given antibody. Expression of various biomarkers in a sample can be analyzed by a number of methodologies, many of which are known in the art and understood by the skilled artisan, including but not limited to, immunohistochemical and/or Western analysis, quantitative blood based assays (as for example Serum ELISA) (to examine, for example, levels of protein expression), biochemical enzymatic activity assays, in situ hybridization, Northern analysis and/or PCR analysis of mRNAs, as well as any one of the wide variety of assays that can be performed by gene and/or tissue array analysis. Typical protocols for evaluating the status of genes and gene products are found, for example in Ausubel et al. eds., 1995, Current Protocols In Molecular Biology, Units 2 (Northern Blotting), 4 (Southern Blotting), 15 (Immunoblotting) and 18 (PCR Analysis). A skilled artisan can readily adapt known protein/antibody detection methods for use in determining whether cells express a marker of the present invention and the relative concentration of that specific polypeptide expression product in blood or other body tissues.

In such alternative methods, a sample may be contacted with an antibody specific for said biomarker under conditions sufficient for an antibody-biomarker complex to form, and then detecting said complex. The presence of the biomarker may be detected in a number of ways, such as by Western blotting, enzyme assays, and ELISA procedures for assaying a wide variety of tissues and samples, including plasma or serum. A wide range of immunoassay techniques using such an assay format are available, see, e.g., U.S. Pat. Nos. 4,016,043, 4,424,279 and 4,018,653. These include both single-site and two-site or “sandwich” assays of the non-competitive types, as well as in the traditional competitive binding assays. These assays also include direct binding of a labeled antibody to a target biomarker.

Sandwich assays are among the most useful and commonly used assays. A number of variations of the sandwich assay technique exist, and all are intended to be encompassed by the present invention. Briefly, in a typical forward assay, an unlabelled antibody is immobilized on a solid substrate, and the sample to be tested brought into contact with the bound molecule. After a suitable period of incubation, for a period of time sufficient to allow formation of an antibody-antigen complex, a second antibody specific to the antigen, labeled with a reporter molecule capable of producing a detectable signal is then added and incubated, allowing time sufficient for the formation of another complex of antibody-antigen-labeled antibody. Any unreacted material is washed away, and the presence of the antigen is determined by observation of a signal produced by the reporter molecule. The results may either be qualitative, by simple observation of the visible signal, or may be quantitated by comparing with a control sample containing known amounts of biomarker.

Variations on the forward assay include a simultaneous assay, in which both sample and labeled antibody are added simultaneously to the bound antibody. These techniques are well known to those skilled in the art, including any minor variations as will be readily apparent. In a typical forward sandwich assay, a first antibody having specificity for the biomarker is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay. The binding processes are well-known in the art and generally consist of cross-linking covalently binding or physically adsorbing, the polymer-antibody complex is washed in preparation for the test sample. An aliquot of the sample to be tested is then added to the solid phase complex and incubated for a period of time sufficient (e.g. 2-40 minutes or overnight if more convenient) and under suitable conditions (e.g. from room temperature to 40° C. such as between 25° C. and 32° C. inclusive) to allow binding of any subunit present in the antibody. Following the incubation period, the antibody subunit solid phase is washed and dried and incubated with a second antibody specific for a portion of the biomarker. The second antibody is linked to a reporter molecule which is used to indicate the binding of the second antibody to the molecular marker.

An alternative method involves immobilizing the target biomarkers in the sample and then exposing the immobilized target to specific antibody which may or may not be labeled with a reporter molecule. Depending on the amount of target and the strength of the reporter molecule signal, a bound target may be detectable by direct labeling with the antibody. Alternatively, a second labeled antibody, specific to the first antibody is exposed to the target-first antibody complex to form a target-first antibody-second antibody tertiary complex. The complex is detected by the signal emitted by the reporter molecule. By “reporter molecule”, as used in the present specification, is meant a molecule which, by its chemical nature, provides an analytically identifiable signal which allows the detection of antigen-bound antibody. The most commonly used reporter molecules in this type of assay are either enzymes, fluorophores or radionuclide containing molecules (i.e. radioisotopes) and chemiluminescent molecules.

In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, -galactosidase and alkaline phosphatase, amongst others. The substrates to be used with the specific enzymes are generally chosen for the production, upon hydrolysis by the corresponding enzyme, of a detectable color change. Examples of suitable enzymes include alkaline phosphatase and peroxidase. It is also possible to employ fluorogenic substrates, which yield a fluorescent product rather than the chromogenic substrates noted above. In all cases, the enzyme-labeled antibody is added to the first antibody-molecular marker complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of biomarker which was present in the sample. Alternately, fluorescent compounds, such as fluorescein and rhodamine, may be chemically coupled to antibodies without altering their binding capacity. When activated by illumination with light of a particular wavelength, the fluorochrome-labeled antibody adsorbs the light energy, inducing a state to excitability in the molecule, followed by emission of the light at a characteristic color visually detectable with a light microscope. As in the EIA, the fluorescent labeled antibody is allowed to bind to the first antibody-molecular marker complex. After washing off the unbound reagent, the remaining tertiary complex is then exposed to the light of the appropriate wavelength, the fluorescence observed indicates the presence of the molecular marker of interest. Immunofluorescence and EIA techniques are both very well established in the art. However, other reporter molecules, such as radioisotope, chemiluminescent or bioluminescent molecules, may also be employed.

Methods of the invention further include protocols which examine the presence and/or expression of mRNAs, in a tissue or cell sample. Methods for the evaluation of mRNAs in cells are well known and include, for example, hybridization assays using complementary DNA probes (such as in situ hybridization using labeled riboprobes, Northern blot and related techniques) and various nucleic acid amplification assays (such as RT-PCR and other amplification type detection methods, such as, for example, branched DNA, SISBA, TMA and the like).

In an embodiment, the level of mRNA corresponding to the marker can be determined both by in situ and by in vitro formats in a biological sample using methods known in the art. Many expression detection methods use isolated RNA. For in vitro methods, any RNA isolation technique that does not select against the isolation of mRNA can be utilized for the purification of RNA from cells. See, e.g., Ausubel et al., Ed., Curr. Prot. Mol. Biol., John Wiley & Sons, NY (1987-1999). Additionally, large numbers of tissue samples can readily be processed using techniques well-known to those of skill in the art, such as, e.g., the single-step RNA isolation process of U.S. Pat. No. 4,843,155. The isolated mRNA can be used in hybridization or amplification assays that include, but are not limited to, Southern or Northern analyses, PCR analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, e.g., a full-length cDNA, or a portion thereof, such as an oligonucleotide of at least 7, 15, 30, 50, 100, 250 or 500 nucleotides in length and sufficient to specifically hybridize under stringent conditions to a mRNA or genomic DNA encoding a marker of the present invention. Other suitable probes for use in the diagnostic assays of the invention are described herein. Hybridization of an mRNA with the probe indicates that the marker in question is being expressed.

In one format, the mRNA is immobilized on a solid surface and contacted with a probe, for example, by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in an Affymetrix gene chip array. A skilled artisan can readily adapt known mRNA detection methods for use in detecting the level of mRNA encoded by the markers of the present invention.

For in situ methods, mRNA does not need to be isolated form the cells prior to detection. In such methods, a cell or tissue sample is prepared/processed using known histological methods. The sample is then immobilized on a support, typically a glass slide, and then contacted with a probe that can hybridize to mRNA that encodes the marker.

As an alternative to making determinations based on the absolute expression level of the marker, determinations may be based on the normalized expression level of the marker. Expression levels are normalized by correcting the absolute expression level of a marker by comparing its expression to the expression of a gene that is not a marker, e.g., a housekeeping gene that is constitutively expressed. Suitable genes for normalization include housekeeping genes, such as the actin gene or epithelial cell-specific genes. This normalization allows the comparison of the expression level in one sample, e.g., a patient sample, to another sample or between samples from different sources.

Alternatively, the expression level can be provided as a relative expression level. To determine a relative expression level of a marker, the level of expression of the marker is determined for 10 or more samples of normal versus disease biological samples, preferably 50 or more samples, prior to the determination of the expression level for the sample in question. The mean expression level of each of the genes assayed in the larger number of samples is determined and this is used as a baseline expression level for the marker. The expression level of the marker determined for the test sample (absolute level of expression) is then divided by the mean expression value obtained for that marker. This provides a relative expression level.

Preferably, the samples used in the baseline determination will be from patients who do not have the polymorphism. The choice of the cell source is dependent on the use of the relative expression level. Using expression found in normal tissues as a mean expression score aids in validating whether the marker assayed is specific (versus normal cells). In addition, as more data is accumulated, the mean expression value can be revised, providing improved relative expression values based on accumulated data.

### Drug Discovery

In other preferred embodiments, the activity of alpha-galactosidase and other biomarkers are useful for the identification of new drugs in the treatment of kidney diseases and disorders. For example, a candidate agent which modulates alpha-galactosidase activity would be an excellent agent for development as a drug in the treatment of diseases associate with abnormal expression or activity of alpha-galactosidase.

Small Molecules: Small molecule test compounds or candidate therapeutic compounds can initially be members of an organic or inorganic chemical library. As used herein, “small molecules” refers to small organic or inorganic molecules of molecular weight below about 3,000 Daltons. The small molecules can be natural products or members of a combinatorial chemistry library. A set of diverse molecules should be used to cover a variety of functions such as charge, aromaticity, hydrogen bonding, flexibility, size, length of side chain, hydrophobicity, and rigidity. Combinatorial techniques suitable for synthesizing small molecules are known in the art, e.g., as exemplified by Obrecht and Villalgordo, Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, for example, Czarnik, Curr. Opin. Chem. Bio., 1:60 (1997). In addition, a number of small molecule libraries are commercially available.

Particular screening applications of this invention relate to the testing of pharmaceutical compounds in drug research. The reader is referred generally to the standard textbook “In vitro Methods in Pharmaceutical Research”, Academic Press, 1997, and U.S. Pat. No. 5,030,015). Assessment of the activity of candidate pharmaceutical compounds generally involves administering a candidate compound, determining any change in the morphology, marker phenotype and expression, or metabolic activity of the cells and function of the cells that is attributable to the compound (compared with untreated cells or cells treated with an inert compound), and then correlating the effect of the compound with the observed change.

The screening may be done, for example, either because the compound is designed to have a pharmacological effect on certain cell types, or because a compound designed to have effects elsewhere may have unintended side effects. Two or more drugs can be tested in combination (by combining with the cells either simultaneously or sequentially), to detect possible drug—drug interaction effects. In some applications, compounds are screened initially for potential toxicity (Castell et al., pp. 375-410 in “In vitro Methods in Pharmaceutical Research,” Academic Press, 1997). Cytotoxicity can be determined in the first instance by the effect on cell viability, survival, morphology, and expression or release of certain markers, receptors or enzymes. Effects of a drug on chromosomal DNA can be determined by measuring DNA synthesis or repair. [³H]thymidine or BrdU incorporation, especially at unscheduled times in the cell cycle, or above the level required for cell replication, is consistent with a drug effect. Unwanted effects can also include unusual rates of sister chromatid exchange, determined by metaphase spread. The reader is referred to A. Vickers (PP 375-410 in “In vitro Methods in Pharmaceutical Research,” Academic Press, 1997) for further elaboration.

In one embodiment of the invention, a method of identifying a candidate agent is provided said method comprising: (a) contacting a biological sample from a patient with the candidate agent and determining the level of expression and/or activity of one or more biomarkers described herein; (b) determining the level of expression and/or activity of a corresponding biomarker or biomarkers in an aliquot of the biological sample not contacted with the candidate agent; (c) observing the effect of the candidate agent by comparing the level of expression and/or activity of the biomarker or biomarkers in the aliquot of the biological sample contacted with the candidate agent and the level of expression and/or activity of the corresponding biomarker or biomarkers in the aliquot of the biological sample not contacted with the candidate agent; and (d) identifying said agent from said observed effect, wherein an at least 1%, 2%, 5%, 10% difference between the level of expression and/or activity of the biomarker or combination of biomarker expression and/or activity in the aliquot of the biological sample contacted with the candidate agent and the level of expression and/or activity of the corresponding biomarker expression and/or activity or combination of biomarker genes in the aliquot of the biological sample not contacted with the candidate agent is an indication of an effect of the candidate agent.

In preferred embodiments, the effects of the drug are correlated with the expression of alpha galactosidase associated with a healthy, normal individual.

In another embodiment of the invention, a candidate agent derived by the method according to the invention is provided.

In another embodiment of the invention, a pharmaceutical preparation comprising an agent according to the invention is provided.

In another preferred embodiment of the invention, a method of producing a drug comprising the steps of the method according to the invention (i) synthesizing the candidate agent identified in step (c) above or an analog or derivative thereof in an amount sufficient to provide said drug in a therapeutically effective amount to a subject; and/or (ii) combining the drug candidate the candidate agent identified in step (c) above or an analog or derivative thereof with a pharmaceutically acceptable carrier.

### Kits:

In another preferred embodiment, a kit comprises primers of alpha-galactosidase; thermostable polymerase, and A, G, C, T nucleotides. The primers can be selected from any nucleic acid region 5′ to the coding region of each molecule, 3′ to the coding region or any region of the molecule selected by one of ordinary skill in the art.

In another preferred embodiment, a kit comprises alpha-galactosidase, peptides thereof, or antibodies specific for alpha-galactosidase or peptides thereof.

In some instances, such as when unusually small amounts of RNA are recovered and only small amounts of cDNA are generated therefrom, it is desirable or necessary to perform a PCR reaction on the first PCR reaction product. That is, if difficult to detect quantities of amplified DNA are produced by the first reaction, a second PCR can be performed to make multiple copies of DNA sequences of the first amplified DNA. A nested set of primers are used in the second PCR reaction. The nested set of primers hybridize to sequences downstream of the 5′ primer and upstream of the 3′ primer used in the first reaction.

According to the invention, diagnostic kits can be assembled which is useful to practice methods of detecting the presence of mRNA or cDNA that encodes alpha-galactosidase in tissue samples. Such diagnostic kits comprise oligonucleotides which are useful as primers for performing PCR methods. It is preferred that diagnostic kits according to the present invention comprise a container comprising a size marker to be run as a standard on a gel used to detect the presence of amplified DNA. The size marker is the same size as the DNA generated by the primers in the presence of the mRNA or cDNA encoding alpha-galactosidase.

In another preferred embodiment, a kit comprises reagents for identifying and measuring the levels of alpha-galactosidase using real-time PCR (RT-PCR). The kit can include one or more of primers suitable for hybridizing to alpha-galactosidase.

Another method of determining whether a sample contains cells expressing alpha-galactosidase, is by Northern blot analysis of mRNA extracted from a tissue sample. The techniques for performing Northern blot analyses are well known by those having ordinary skill in the art and are described in Sambrook, J. et al., Molecular Cloning: A Laboratory Manual, (1989) Cold Spring Harbor Laboratory Press, Cold Spring Harbor, N.Y. mRNA extraction, electrophoretic separation of the mRNA, blotting, probe preparation and hybridization are all well known techniques that can be routinely performed using readily available starting material.

One having ordinary skill in the art, performing routine techniques, could design probes to identify mRNA encoding alpha-galactosidase, using their sequence information. The mRNA is extracted using poly dT columns and the material is separated by electrophoresis and, for example, transferred to nitrocellulose paper. Labeled probes made from an isolated specific fragment or fragments can be used to visualize the presence of a complementary fragment fixed to the paper.

According to the invention, diagnostic kits can be assembled which is useful to practice methods of detecting the presence of mRNA that encodes alpha-galactosidase in tissue samples by Northern blot analysis. Such diagnostic kits comprise oligonucleotides which are useful as probes for hybridizing to the mRNA. The probes may be radio labeled. It is preferred that diagnostic kits according to the present invention comprise a container comprising a size marker to be run as a standard on a gel. It is preferred that diagnostic kits according to the present invention comprise a container comprising a positive control which will hybridize to the probe.

Another method of detecting the presence of mRNA encoding alpha-galactosidase is by oligonucleotide hybridization technology. Oligonucleotide hybridization technology is well known to those having ordinary skill in the art. Briefly, detectable probes which contain a specific nucleotide sequence that will hybridize to nucleotide sequence of mRNA encoding alpha-galactosidase. RNA or cDNA made from RNA from a sample is fixed, usually to filter paper or the like. The probes are added and maintained under conditions that permit hybridization only if the probes fully complement the fixed genetic material. The conditions are sufficiently stringent to wash off probes in which only a portion of the probe hybridizes to the fixed material. Detection of the probe on the washed filter indicates complementary sequences. One having ordinary skill in the art, using the sequence information of alpha-galactosidase can design probes which are fully complementary to mRNA sequences but not genomic DNA sequences. Hybridization conditions can be routinely optimized to minimize background signal by non-fully complementary hybridization.

The present invention includes labeled oligonucleotides which are useful as probes for performing oligonucleotide hybridization. That is, they are fully complementary with mRNA sequences but not genomic sequences. For example, the mRNA sequence includes portions encoded by different exons. The labeled probes of the present invention are labeled with radiolabeled nucleotides or are otherwise detectable by readily available nonradioactive detection systems.

According to the invention, diagnostic kits can be assembled which is useful to practice oligonucleotide hybridization methods of the invention. Such diagnostic kits comprise a labeled oligonucleotide which encodes portions of at least one of alpha-galactosidase encoded by different exons. It is preferred that labeled probes of the oligonucleotide diagnostic kits according to the present invention are labeled with a radio nucleotide. The oligonucleotide hybridization-based diagnostic kits according to the invention preferably comprise DNA samples that represent positive and negative controls. A positive control DNA sample is one that comprises a nucleic acid molecule which has a nucleotide sequence that is fully complementary to the probes of the kit such that the probes will hybridize to the molecule under assay conditions. A negative control DNA sample is one that comprises at least one nucleic acid molecule, the nucleotide sequence of which is partially complementary to the sequences of the probe of the kit. Under assay conditions, the probe will not hybridize to the negative control DNA sample.

Another aspect of the invention relates to methods of analyzing tissue samples which are fixed sections routinely prepared by surgical pathologists to characterize and evaluate cells. In some embodiments, the cells are from kidney tissue and are analyzed to determine and evaluate the extent of alpha-galactosidase expression.

The present invention relates to in vitro kits for evaluating tissues samples to determine the level of alpha-galactosidase expression and to reagents and compositions useful to practice the same. The tissue is analyzed to identify the presence or absence of the at least one of alpha-galactosidase protein. Techniques such as binding assays, enzymatic assays and immunohistochemistry assays may be performed to determine whether at least alpha-galactosidase is absent in cells in the tissue sample. The presence of mRNA that encodes the at least one of alpha-galactosidase protein or cDNA generated therefrom can be determined using techniques such as in situ hybridization, immunohistochemistry.

In situ hybridization technology is well known by those having ordinary skill in the art. Briefly, cells are fixed and detectable probes which contain a specific nucleotide sequence are added to the fixed cells. If the cells contain complementary nucleotide sequences, the probes, which can be detected, will hybridize to them. One having ordinary skill in the art, using the sequence information of alpha-galactosidase can design probes useful in in situ hybridization technology to identify cells that express alpha-galactosidase.

For in situ hybridization according to the invention, it is preferred that the probes are detectable by fluorescence. A common procedure is to label probe with biotin-modified nucleotide and then detect with fluorescently-tagged avidin. Hence, the probe does not itself have to be labeled with florescent but can be subsequently detected with florescent marker.

Cells are fixed and the probes are added to the genetic material. Probes will hybridize to the complementary nucleic acid sequences present in the sample. Using a fluorescent microscope, the probes can be visualized by their fluorescent markers.

According to the invention, diagnostic kits can be assembled which is useful to practice in situ hybridization methods of the invention are fully complementary with mRNA sequences but not genomic sequences. For example, the mRNA sequence includes portions encoded by different exons. It is preferred that labeled probes of the in situ diagnostic kits according to the present invention are labeled with a fluorescent marker.

Immunohistochemistry techniques may be used to identify and essentially stain cells which express alpha-galactosidase. Anti-marker, e.g. anti-alpha-galactosidase antibodies are contacted with fixed cells and the alpha-galactosidase present in the cells reacts with the antibodies. The antibodies are detectably labeled or detected using labeled second antibody or protein A to stain the cells.

According to some embodiments, diagnostic reagents and kits are provided for performing immunoassays to determine the presence or absence of alpha-galactosidase protein or fragments thereof in a sample from an individual. Kits may additionally include one or more of the following: means for detecting antibodies bound to alpha-galactosidase present in a sample, instructions for performing the method, and diagrams or photographs that are representative of how positive and/or negative results appear. In addition, kits may comprise optional positive controls such as alpha-galactosidase protein. Further, optional negative controls may be provided.

Immunoassay methods may be used to identify individuals identifying and measuring the levels of alpha-galactosidase by detecting the absence or deficiency of alpha-galactosidase in sample of tissue or body fluid using antibodies which bind to alpha-galactosidase. The antibodies are preferably monoclonal antibodies. The antibodies are preferably raised against alpha-galactosidase made in human cells. Immunoassays are well known and there design may be routinely undertaken by those having ordinary skill in the art. The techniques for producing monoclonal antibodies are outlined in Harlow, E. and D. Lane (1988) Antibodies: A Laboratory Manual, Cold Spring Harbor Laboratory, Cold Spring Harbor, N.Y., which is incorporated herein by reference, provide detailed guidance for the production of hybridomas and monoclonal antibodies which specifically bind to alpha-galactosidase.

According to some embodiments, immunoassays comprise allowing proteins in the sample to bind a solid phase support such as a plastic surface. Detectable antibodies are then added which selectively binding to alpha-galactosidase. Detection of the detectable antibody indicates the presence alpha-galactosidase. The detectable antibody may be a labeled or an unlabelled antibody. Unlabelled antibody may be detected using a second, labeled antibody that specifically binds to the first antibody or a second, unlabelled antibody which can be detected using labeled protein A, a protein that complexes with antibodies. Various immunoassay procedures are described in Immunoassays for the 80's, Voller, et al., Ed., University Park, 1981, which is incorporated herein by reference.

Simple immunoassays may be performed in which a solid phase support is contacted with the test sample. Any proteins present in the test sample bind the solid phase support and can be detected by a specific, detectable antibody preparation. Such a technique is the essence of the dot blot, Western blot and other such similar assays.

Other immunoassays may be more complicated but actually provide excellent results. Typical and preferred immunometric assays include “forward” assays for the detection of a protein in which a first anti-protein antibody bound to a solid phase support is contacted with the test sample. After a suitable incubation period, the solid phase support is washed to remove unbound protein. A second, distinct anti-protein antibody is then added which is specific for a portion of the specific protein not recognized by the first antibody. The second antibody is preferably detectable. After a second incubation period to permit the detectable antibody to complex with the specific protein bound to the solid phase support through the first antibody, the solid phase support is washed a second time to remove the unbound detectable antibody. Alternatively, the second antibody may not be detectable. In this case, a third detectable antibody, which binds the second antibody, is added to the system. This type of “forward sandwich” assay may be a simple yes/no assay to determine whether binding has occurred or may be made quantitative by comparing the amount of detectable antibody with that obtained in a control. Such “two-site” or “sandwich” assays are described by Wide, Radioimmune Assay Method, (1970) Kirkham, Ed., E. & S. Livingstone, Edinburgh, pp. 199-206, which is incorporated herein by reference.

Other types of immunometric assays are the so-called “simultaneous” and “reverse” assays. A simultaneous assay involves a single incubation step wherein the first antibody bound to the solid phase support, the second, detectable antibody and the test sample are added at the same time. After the incubation is completed, the solid phase support is washed to remove unbound proteins. The presence of detectable antibody associated with the solid support is then determined as it would be in a conventional “forward sandwich” assay. The simultaneous assay may also be adapted in a similar manner for the detection of antibodies in a test sample.

The “reverse” assay comprises the stepwise addition of a solution of detectable antibody to the test sample followed by an incubation period and the addition of antibody bound to a solid phase support after an additional incubation period. The solid phase support is washed in conventional fashion to remove unbound protein/antibody complexes and unreacted detectable antibody. The determination of detectable antibody associated with the solid phase support is then determined as in the “simultaneous” and “forward” assays. The reverse assay may also be adapted in a similar manner for the detection of antibodies in a test sample.

The “alpha-galactosidase” marker is used throughout as merely exemplary and does not limit the description or embodiments to just alpha-galactosidase, fragments, mutants, variants and complementary sequences thereof. The first component of the immunometric assay may be added to nitrocellulose or other solid phase support which is capable of immobilizing proteins. The first component for determining the presence of alpha-galactosidase in a test sample is anti-alpha-galactosidase antibody. By “solid phase support” or “support” is intended any material capable of binding proteins. Well-known solid phase supports include glass, polystyrene, polypropylene, polyethylene, dextran, nylon, amylases, natural and modified celluloses, polyacrylamides, agaroses, and magnetite. The nature of the support can be either soluble to some extent or insoluble for the purposes of the present invention. The support configuration may be spherical, as in a bead, or cylindrical, as in the inside surface of a test tube or the external surface of a rod. Alternatively, the surface may be flat such as a sheet, test strip, etc. Those skilled in the art will know many other suitable “solid phase supports” for binding proteins or will be able to ascertain the same by use of routine experimentation. A preferred solid phase support is a 96-well microtiter plate.

To detect the presence of alpha-galactosidase, detectable anti-alpha-galactosidase antibodies are used. Several methods are well known for the detection of antibodies. One method in which the antibodies can be detectably labeled is by linking the antibodies to an enzyme and subsequently using the antibodies in an enzyme immunoassay (EIA) or enzyme-linked immunosorbent assay (ELISA), such as a capture ELISA. The enzyme, when subsequently exposed to its substrate, reacts with the substrate and generates a chemical moiety which can be detected, for example, by spectrophotometric, fluorometric or visual means. Enzymes which can be used to detectably label antibodies include, but are not limited to malate dehydrogenase, staphylococcal nuclease, delta-5-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. One skilled in the art would readily recognize other enzymes which may also be used.

Another method in which antibodies can be detectably labeled is through radioactive isotopes and subsequent use in a radioimmunoassay (RIA) (see, for example, Work, et al., Laboratory Techniques and Biochemistry in Molecular Biology, North Holland Publishing Company, N.Y., 1978, which is incorporated herein by reference). The radioactive isotope can be detected by such means as the use of a gamma counter or a scintillation counter or by autoradiography. Isotopes which are particularly useful for the purpose of the present invention are ³H, ¹²⁵I, ¹³¹I, ³⁵S, and ¹⁴C. One skilled in the art would readily recognize other radioisotopes which may also be used.

It is also possible to label the antibody with a fluorescent compound. When the fluorescent-labeled antibody is exposed to light of the proper wavelength, its presence can be detected due to its fluorescence. Among the most commonly used fluorescent labeling compounds are fluorescein isothiocyanate, rhodamine, phycoerythrin, phycocyanin, allophycocyanin, o-phthaldehyde and fluorescamine. One skilled in the art would readily recognize other fluorescent compounds which may also be used.

Antibodies can also be detectably labeled using fluorescence-emitting metals such as ¹⁵²Eu, or others of the lanthanide series. These metals can be attached to the protein-specific antibody using such metal chelating groups as diethylenetriaminepentaacetic acid (DTPA) or ethylenediamine-tetraacetic acid (EDTA). One skilled in the art would readily recognize other fluorescence-emitting metals as well as other metal chelating groups which may also be used.

Antibodies can also be detectably labeled by coupling to a chemiluminescent compound. The presence of the chemiluminescent-labeled antibody is determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester. One skilled in the art would readily recognize other chemiluminescent compounds which may also be used.

Likewise, a bioluminescent compound may be used to label antibodies. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin. One skilled in the art would readily recognize other bioluminescent compounds which may also be used.

Detection of the protein-specific antibody, fragment or derivative may be accomplished by a scintillation counter if, for example, the detectable label is a radioactive gamma emitter. Alternatively, detection may be accomplished by a fluorometer if, for example, the label is a fluorescent material. In the case of an enzyme label, the detection can be accomplished by colorometric methods which employ a substrate for the enzyme. Detection may also be accomplished by visual comparison of the extent of enzymatic reaction of a substrate in comparison with similarly prepared standards. One skilled in the art would readily recognize other appropriate methods of detection which may also be used.

The binding activity of a given lot of antibodies may be determined according to well known methods. Those skilled in the art will be able to determine operative and optimal assay conditions for each determination by employing routine experimentation.

In a preferred embodiment, a kit comprises a filter paper and means to detect the presence or absence of alpha-galactosidase in a sample as described in detail in the examples section which follows.

In a preferred embodiment, the kits include antibodies for detection of alpha-galactosidase and identifying variants thereof, using Western blot analysis.

Positive and negative controls may be performed in which known amounts of alpha-galactosidase and no alpha-galactosidase, respectively, are added to assays being performed in parallel with the test assay. One skilled in the art would have the necessary knowledge to perform the appropriate controls.

An “antibody composition” refers to the antibody or antibodies required for the detection of the protein. For example, the antibody composition used for the detection of alpha-galactosidase in a test sample comprises a first antibody which binds alpha-galactosidase, as well as a second or third detectable antibody that binds the first or second antibody, respectively.

To examine a test sample for the presence or absence of alpha-galactosidase, a standard immunometric assay such as the one described herein may be performed. A first anti-alpha-galactosidase antibody, which recognizes, for example, a specific portion of alpha-galactosidase is added to a 96-well microtiter plate in a volume of buffer. The plate is incubated for a period of time sufficient for binding to occur and subsequently washed with PBS to remove unbound antibody. The plate is then blocked with a PBS/BSA solution to prevent sample proteins from non-specifically binding the microtiter plate. Test sample are subsequently added to the wells and the plate is incubated for a period of time sufficient for binding to occur. The wells are washed with PBS to remove unbound protein. Labeled anti-alpha-galactosidase antibodies, which recognize portions of alpha-galactosidase not recognized by the first antibody, are added to the wells. The plate is incubated for a period of time sufficient for binding to occur and subsequently washed with PBS to remove unbound, labeled anti-alpha-galactosidase antibody. The amount of labeled and bound anti-alpha-galactosidase antibody is subsequently determined by standard techniques.

Kits which are useful for the detection of, for example, alpha-galactosidase in a test sample comprise a container comprising anti-alpha-galactosidase antibodies and a container or containers comprising controls. Controls include one control sample which does not contain alpha-galactosidase and/or another control sample which contained alpha-galactosidase. The anti-alpha-galactosidase antibodies used in the kit are detectable such as being detectably labeled. If the detectable anti-alpha-galactosidase antibody is not labeled, it may be detected by second antibodies or protein A, for example, which may also be provided in some kits in separate containers. Additional components in some kits include solid support, buffer, and instructions for carrying out the assay. The immunoassay is useful for detecting alpha-galactosidase in homogenized tissue samples and body fluid samples including the plasma portion or cells in the fluid sample.

As discussed infra, in all of the embodiments, the detection can also include other antibodies for example, as a control, as part of the detection and the like.

Western blots may be used in methods of identifying individuals with, for example, early stage CDK and other kidney disorders, by quantifying alpha-galactosidase in samples of tissue, such as for example, kidney. Western blots use detectable anti-alpha-galactosidase antibodies to bind to any alpha-galactosidase present in a sample and thus indicate the presence of the protein in the sample. Western blot techniques, which are described in Sambrook, J. et al., (1989) Molecular Cloning: A Laboratory Manual, Cold Spring Harbor Laboratory Press, Cold Spring Harbor, N.Y., which is incorporated herein by reference, are similar to immunoassays with the essential difference being that prior to exposing the sample to the antibodies, the proteins in the samples are separated by gel electrophoresis and the separated proteins are then probed with antibodies. In some preferred embodiments, the matrix is an SDS-PAGE gel matrix and the separated proteins in the matrix are transferred to a carrier such as filter paper prior to probing with antibodies.

Anti-alpha-galactosidase antibodies described above are useful in Western blot methods. Generally, samples are homogenized and cells are lysed using detergent such as Triton-X. The material is then separated by the standard techniques in Sambrook, J. et al., (1989) Molecular Cloning: A Laboratory Manual, Cold Spring Harbor Laboratory Press, Cold Spring Harbor, N.Y.

Kits which are useful for the detection of alpha-galactosidase in a test sample by Western blot comprise a container comprising anti-alpha-galactosidase antibodies and a container or containers comprising controls. For example, controls include one control sample which does not contain alpha-galactosidase and/or another control sample which contained alpha-galactosidase. The anti-alpha-galactosidase antibodies used in the kit are detectable such as being detectably labeled. If the detectable anti-alpha-galactosidase is not labeled, it may be detected by second antibodies or protein A for example which may also be provided in some kits in separate containers. Additional components in some kits include instructions for carrying out the assay. The means to detect anti-alpha-galactosidase antibodies that are bound to alpha-galactosidase include the immunoassays described above.

Aspects of the present invention also include various methods of determining whether a sample contains cells that express alpha-galactosidase by sequence-based molecular analysis. Several different methods are available for doing so including those using Polymerase Chain Reaction (PCR) technology, using Northern blot technology, oligonucleotide hybridization technology, and in situ hybridization technology. According to the invention, samples are screened to determine the presence or absence of mRNA that encodes alpha-galactosidase.

The invention also relates to oligonucleotide probes and primers used in the methods of identifying mRNA that encodes alpha-galactosidase complex and to diagnostic kits which comprise such components. The mRNA sequence-based methods for determining whether a sample mRNA encoding alpha-galactosidase include but are not limited to PCR technology, Northern and Southern blot technology, in situ hybridization technology and oligonucleotide hybridization technology.

While various embodiments of the present invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the present invention should not be limited by any of the above described embodiments.

All documents mentioned herein are incorporated herein by reference. All publications and patent documents cited in this application are incorporated by reference for all purposes to the same extent as if each individual publication or patent document were so individually denoted. By their citation of various references in this document, Applicants do not admit any particular reference is “prior art” to their invention. Embodiments of inventive compositions and methods are illustrated in the following examples.

### EXAMPLES

The following non-limiting Examples serve to illustrate selected embodiments of the invention. It will be appreciated that variations in proportions and alternatives in elements of the components shown will be apparent to those skilled in the art and are within the scope of embodiments of the present invention.

### Example 1

### Dried Blood Spot Testing

This study was set up to determine if dried blood spot testing can be reliably used to detect alpha-GAL levels in healthy control patients and in patients with symptoms of Fabry disease and to explore and validate the use of dried blood spot (DBS) method as a tool for detecting alpha galactosidase A activity in patients with renal disease.

Subject Selection: The subspecialty clinics are ideal sites to locate potential carriers for enzyme deficiency in alpha Galactosidase A. Subjects were identified according to the outlined target populations by their physicians from among the patients of the physician investigator(s), and made aware of the study. One of the study staff (subject's physician investigator, when applicable) approaches the subject and explains to the subject the rationale for the study and for consent. Consent was obtained from the subject by physician investigator (when applicable). Sufficient time was given to subjects to consider participation providing ample time for discussion and consultation with family members or their physicians. If there was any question that the subject was unable to comprehend the informed consent process, then this subject was not used for the study. Investigators reinforced with their patients that participation was voluntary, that they did not have to participate, and the decision not to participate would not affect their care, presently or in the future.

A total of 200 subjects were selected for the study (50 subjects with proteinuria and 50 controls). Male and female patients in the age group of 18-75 were considered for this study. Inclusion criteria for study control group: Target Populations: clinic patients (male and female)-Normal renal function (GFR>100 ml/min), no significant proteinuria (<300 mg/day), normal cardiac function and no history of CVA. Inclusion criteria for study test group: Renal: Target Populations:
a) clinic patients (male and female)
   Normal renal function (GFR>100 ml/min), proteinuria (>300 mg/day)
   CKD stage 2-5 (GFR between 20 and 90 ml/min), proteinuria (>300 mg/day)
   Dialysis patients with unexplained etiology of renal failure
   Unexplained left ventricular hypertrophy (Echo, EKG) in the absence of HTN
   Premature coronary artery disease or Congestive heart failure (<50 years age)
   Unexplained conduction abnormalities, Arrhythmias
   Strokes of unknown origin including TIA (<50 years age)

No particular ethnic group was excluded or disproportionately burdened. Women and minorities were enrolled in proportion to their representation as subjects cared for at the institution. There was no remuneration for this study.

Confidentiality is maintained at all times. Patients were assigned specific numbers to maintain confidentiality and patient-specific identifiers were not recorded on data collection sheets which were submitted to sponsors or other sites. At completion of the study all identifiers were removed. During the study only study staff had access to subject information. The identifiers were maintained on a password-protected computer which only study staff have access to.

Study Procedures

Study visits and Parameters to be measured: No special study visit is required. Blood spot sample from the subjects was collected at the time of their visit to their physician investigator's office. Blood spots were transferred on a Whatman filter paper and safely stored refrigerated. Every 2 weeks blood spot samples were used for alpha-GAL enzymatic activity testing using the standard laboratory protocol of Chamoles and coworkers (Clin Chim Acta 2001; 308:195-6).

Data to be collected/When data to be collected: Subject data was collected at the time of the study visit. Blood spots were stored refrigerated and enzyme analyses were performed every 2 weeks. Alpha-GAL activity was assayed using a fluorometer which can quantify and detect cleavage of an artificial fluorogenic alpha-GAL substrate.

Biostatistical Analysis: The study was an exploratory study to find out if dried blood spot testing can reliably detect the activity of alpha-GAL enzyme. Thus, a large number of patients (200) were analyzed. It is difficult to estimate the appropriate sample size using simple methods for recommending sample sizes. Impact of some other variables (natural variations in phenotype), such as subject's age, length of diagnosis, previous medications and others, cannot be reliably assessed at this time. It is expected that the results from this study will help to better estimate the sample size requirements for a follow-on validation study with a higher number of patients if necessary.

Specific data variables being collected for the study: The study was conducted on subjects diagnosed with proteinuria and normal renal function, proteinuria with chronic kidney disease (CKD), heart failure and stroke patients (see inclusion criteria above). A control population of healthy subjects was used in this study (see inclusion criteria above). Specific data variables are the values of the alpha-Gal activity being detected. The enzyme activity values are used to calculate Coefficient of variation (CV) (standard deviation (SD)/Mean×100) across all the samples.

Study end points: This was an exploratory study to investigate the feasibility of using whole dried blood spot as a tool to detect Fabry disease.

Statistical methods: It hypothesized that a number of enzymatic activities would be differentially present in healthy individuals (control) and in some of the patients presenting with symptoms seen in Fabry disease. The development of statistical algorithms for enzyme activities is an active area of research. One simple approach is to consider only enzyme activities that are differentially present 2-fold or higher (>2 fold or <0.5 fold in test samples vs. the controls) with t-test p-values <0.01 across all the test samples. An alternative method would be to ascertain how well it distinguishes between case and control subjects. If an enzymatic activity is measured on a binary scale (positive versus negative), the true-positive rate (TPR), i.e., the proportion of case subjects who are biomarker positive, and the false-positive rate (FPR), i.e., the proportion of control subjects who are biomarker positive, summarize its ability to discriminate between disease and non-disease. Sensitivity and specificity are commonly used terms for TPR and 1-FPR. If an enzymatic activity result can take many values, with larger values, for example, being more strongly indicative of disease, a receiver operating characteristic (ROC) curve is used. Advantages of the ROC curve over simple frequencies and summary statistics for enzyme activities data are (a) that it does not depend on the scale of raw-data measurements, which greatly facilitates comparison of the discriminatory capacities of different enzyme activities; and (b) that it displays true- and false-positive rates, quantities that are more relevant for screening purposes than the enzyme activity values themselves. Since the low false-positive rates are of interest for screening, the portion of the ROC curve relating to low FPRs would be the focus of data analysis. One simple approach is to rank the enzymatic activity values on the basis of a summary statistic, such as the area under the ROC curve [or under that part pertaining to low FPRs or other restricted region] and to select those that rank highest.

Power analysis (sample size, evaluable subjects): As described above, it is difficult to effectively estimate sample size for an exploratory study of this nature. However, as a starting point, a sample set of at least 50 from each group was used (in order to do multivariate analysis and to give >90% statistical confidence in a single marker with p values <0.01). Ten samples from each group was also collected for preliminary confirmatory/validation studies. Results were analyzed using MatLab and R statistical package.

Monitoring and Quality Assurance: Subjects are in hospital at time of blood collection with close monitoring. All data is kept in the Nephrology database. All adverse events are reported to the IRB as per HRC guidelines. All testing is documented in appropriate laboratory notebooks.

Results:

The α-galactosidase A activity assay from dry blood samples was successfully set up. The procedure requires a small volume of blood and has been tightly regulated to support a reliable and safe specimen collection and delivery system at a significantly low cost than any currently available assay. The assay is sensitive enough to pick up low levels of a-galactosidase A activity. The relevant statistical data analyses yielded high precision, i.e., low inter-plate and intra-plate variability.

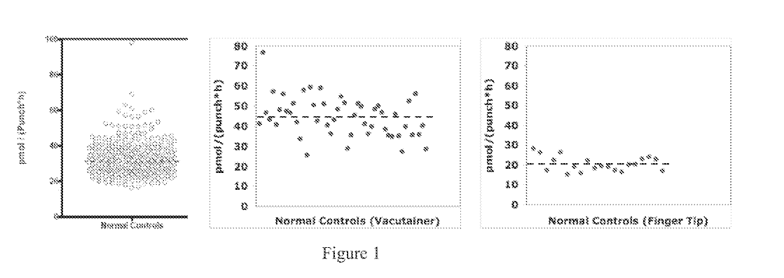

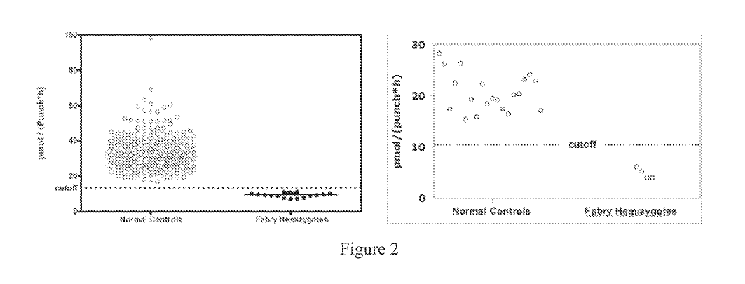

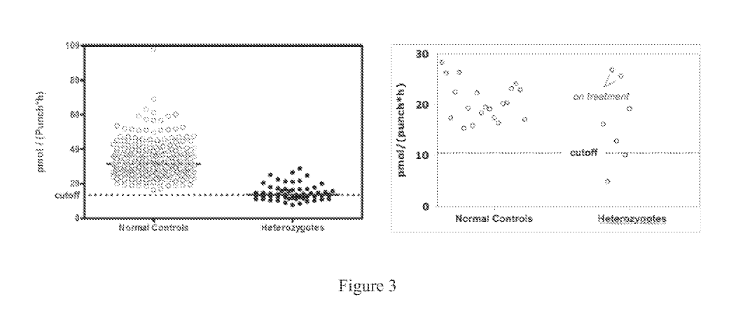

The assay was validated by using 70 normal control (50 vacutainer and 20 finger tip) samples (FIG. 1) and 11 clinical (4 hemizygotes and 7 heterozygotes) samples (FIGS. 2 and 3) and comparing the critical data (for standard curve, cutoff value for the enzyme activity in diagnosing Fabry disease, etc.) with the data supplied by Genzyme.

### Example 2

### Determination of Alpha-Galactosidase Activity for Screening of Fabry Disease and Correlation with CKD Stages Using Ambulatory Monospot Blood Testing

Partial or complete deficiency of alpha-GAL leads to progressive accumulation of glycosphingolipids, particularly globotriaosylceramide (GL-3), in visceral tissues and the vascular endothelium throughout the body leading to multi-systemic damage to the kidney, heart, and cerebrovascular system. Blood activity levels of alpha galactosidase (alpha-Gal) are usually measured in the context of diagnosing Fabry disease but this test is not readily available in hospitals or physician practice. To evaluate the feasibility of an ambulatory test system, the study was performed to validate whether dried blood spot testing can be reliably used to detect alpha-GAL levels in healthy control patients and in patients with symptoms of Fabry disease (instead of the routine more expensive and invasive venous blood sampling technique). After reliable set up of the testing system following the protocol by Nestor Chamoles (Clin Chim Acta 2001; 308:195-6), the relationship of alpha-GAL activity levels in patients was analyzed with different stages of Chronic Kidney Disease (CKD 2-5).

Method: For the validation study cases were identified from the outpatient clinic at Massachusetts General Hospital, Boston. Control blood was obtained from healthy volunteers or from commercial sources. For the correlation study, patients with renal failure (CKD 2—60<GFR<89, CKD 3—30<GFR<59, CKD 4—15<GFR<29, CKD 5—GFR<15) were identified from the renal outpatient clinic at Jackson Memorial Hospital at University of Miami.

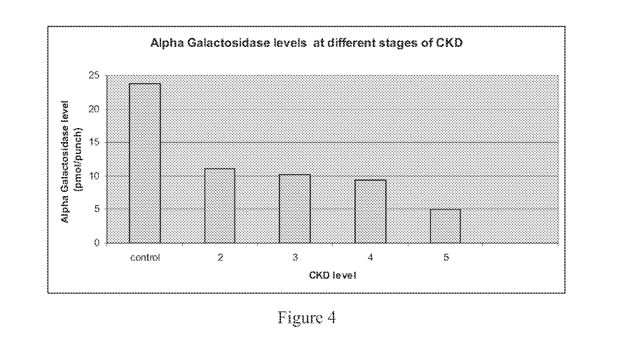

Results: The validation study involved 4 patients with Fabry's disease, 7 known carriers and 51 controls. The median values of Alpha-Gal in the three groups were 4.6 pmol/punch, 16.2 pmol/punch and 42 pmol/punch respectively. There was a significant difference (P=***) between the controls and patients with Fabry's disease. Secondly, values of Alpha-Gal decreased progressively as the level of renal impairment (determined by CKD stage) declined (FIG. 4).

Conclusion:

i) Fingerprick Testing can successfully and reliably be used to measure Alpha Gal levels and therefore enable a less invasive method for the screening of Fabry's Disease.

ii) Alpha Gal enzyme activity decreases with declining renal function. Further studies will be conducted to understand the etiology of this yet undetermined phenomenon.

| Patient | CKD2 | pmol/punch * h |
|---|---|---|
| 4 | 1 | 4.7 |
| 44 | 2 | 13.8 |
| 48 | 3 | 18.0 |
| 58 | 4 | 8.2 |
| 63 | 5 | 8.8 |
| 91 | 6 | 9.5 |
| 110 | 7 | 11.2 |
| 111 | 8 | 19.6 |
| 138 | 9 | 15.4 |
| 143 | 10 | 10.0 |
| 147 | 11 | 11.4 |
| 148 | 12 | 11.1 |
| 149 | 13 | 6.9 |
| 151 | 14 | 14.0 |
| 152 | 15 | 15.1 |
| 134 | 16 | 4.3 |
| | Median | 11.1 |
| Patient | CKD3 | pmol/punch * h |
| 1 | 1 | 7.8 |
| 2 | 2 | 6.8 |
| 3 | 3 | 21.0 |
| 5 | 4 | 14.4 |
| 6 | 5 | 12.0 |
| 8 | 6 | 10.9 |
| 9 | 7 | 11.0 |
| 10 | 8 | 8.0 |
| 11 | 9 | 21.5 |
| 26 | 10 | 12.4 |
| 28 | 11 | 15.3 |
| 29 | 12 | 11.4 |
| 32 | 13 | 12.7 |
| 33 | 14 | 4.4 |
| 34 | 15 | 14.2 |
| 37 | 16 | 12.0 |
| 43 | 17 | 3.7 |
| 46 | 18 | 6.7 |
| 47 | 19 | 4.9 |
| 55 | 20 | 11.1 |
| 59 | 21 | 9.7 |
| 60 | 22 | 3.8 |
| 62 | 23 | 2.9 |
| 64 | 24 | 9.2 |
| 79 | 25 | 9.9 |
| 80 | 26 | 4.8 |
| 86 | 27 | 17.0 |
| 90 | 28 | 4.1 |
| 92 | 29 | 7.2 |
| 94 | 30 | 5.4 |
| 95 | 31 | 7.7 |
| 98 | 32 | 11.3 |
| 99 | 33 | 3.8 |
| 109 | 34 | 9.1 |
| 112 | 35 | 2.3 |
| 137 | 36 | 6.0 |
| 139 | 37 | 7.9 |
| 140 | 38 | 10.7 |
| 141 | 39 | 3.1 |
| 144 | 40 | 12.8 |
| 145 | 41 | 16.6 |
| 146 | 42 | −0.5 |
| 113 | 43 | 9.3 |
| 114 | 44 | 10.7 |
| 115 | 45 | 10.5 |
| 117 | 46 | 9.5 |
| 119 | 47 | 10.8 |
| 120 | 48 | 15.0 |
| 122 | 49 | 18.2 |
| 123 | 50 | 6.2 |
| 124 | 51 | 15.7 |
| 125 | 52 | 13.7 |
| 128 | 53 | 4.5 |
| 129 | 54 | 14.2 |
| 132 | 55 | 11.5 |
| 133 | 56 | 5.7 |
| 135 | 57 | 2.7 |
| 30 | 58 | 10.7 |
| 40 | 59 | 10.6 |
| 154 | 60 | 14.0 |
| | Median | 10.2 |
| Patient | CKD4 | pmol/punch * h |
| 13 | 1 | 23.8 |
| 14 | 2 | 6.2 |
| 25 | 3 | 15.4 |
| 27 | 4 | 7.8 |
| 45 | 5 | 10.3 |
| 49 | 6 | 9.4 |
| 51 | 7 | 3.5 |
| 53 | 8 | −1.6 |
| 56 | 9 | 11.8 |
| 150 | 10 | 8.6 |
| 116 | 11 | 3.5 |
| 118 | 12 | 11.1 |
| 127 | 13 | 9.9 |
| 21 | 14 | 9.5 |
| | Median | 9.4 |
| Patient | CKD5 | pmol/punch * h |
| 7 | 1 | 8.1 |
| 12 | 2 | 11.6 |
| 15 | 3 | 5.7 |
| 16 | 4 | 1.7 |
| 17 | 5 | 4.3 |
| 18 | 6 | 19.5 |
| 19 | 7 | 29.8 |
| 20 | 8 | 35.5 |
| 21 | 9 | 13.4 |
| 22 | 10 | 5.0 |
| 23 | 11 | 6.3 |
| 24 | 12 | 13.1 |
| 31 | 13 | 24.7 |
| 35 | 14 | 8.7 |
| 36 | 15 | 12.5 |
| 38 | 16 | 6.8 |
| 42 | 17 | 2.5 |
| 50 | 18 | 0.4 |
| 52 | 19 | 3.5 |
| 54 | 20 | −0.8 |
| 61 | 21 | −1.6 |
| 65 | 22 | 4.3 |
| 66 | 23 | 5.9 |
| 67 | 24 | −0.4 |
| 68 | 25 | 6.4 |
| 69 | 26 | 1.0 |
| 70 | 27 | 7.3 |
| 71 | 28 | 1.7 |
| 72 | 29 | 9.2 |
| 73 | 30 | −0.1 |
| 74 | 31 | −0.4 |
| 75 | 32 | 5.3 |
| 77 | 33 | 0.7 |
| 78 | 34 | 3.8 |
| 81 | 35 | 3.6 |
| 83 | 36 | 39.5 |
| 84 | 37 | −0.2 |
| 85 | 38 | 1.1 |
| 88 | 39 | 5.6 |
| 89 | 40 | 8.7 |
| 96 | 41 | 3.3 |
| 97 | 42 | 11.2 |
| 104 | 43 | −0.1 |
| 105 | 44 | 17.3 |
| 106 | 45 | 8.8 |
| 107 | 46 | 9.0 |
| 108 | 47 | 0.5 |
| 121 | 48 | 3.8 |
| 126 | 49 | 12.8 |
| 130 | 50 | 1.2 |
| 131 | 51 | −2.0 |
| 103 | 52 | 4.1 |
| 101 | 53 | 3.8 |
| | Median | 5.0 |

| | | Miami | Boston |
|---|---|---|---|
| Patient | Fabry | pmol/punch * h | pmol/punch * h |
| 1.2 | 1 | 0.2 | 6.1 |
| 1.6 | 2 | 7.1 | 5.2 |
| 1.12 | 3 | 7.3 | 4.0 |
| 1.14 | 4 | 6.7 | 4 |
| | Median | 6.9 | 4.6 |
| | | Miami | Boston |
| Patient | Carrier | pmol/punch * h | pmol/punch * h |
| 1.1 | 1 | 6.5 | 16.2 |
| 1.4 | 2 | 5 | 4.9 |
| 1.5 | 3 | 19.4 | 26.8 |
| 1.7 | 4 | 5 | 12.9 |
| 1.8 | 5 | 11.7 | 25.6 |
| 1.11 | 6 | 0.9 | 10.2 |
| 1.13 | 7 | 10.3 | 19.2 |
| | Median | 6.5 | 16.2 |

| | | Miami | Boston |
|---|---|---|---|
| Patient | Unknown | pmol/punch * h | pmol/punch * h |
| 1.3 | 1 | 12.6 | 28.3 |
| 1.9 | 2 | 13.9 | 26.2 |
| 1.10 | 3 | 8 | 17.4 |
| | Median | 12.6 | 26.2 |

| | | Miami | Boston |
|---|---|---|---|
| Patient | Control | pmol/punch * h | pmol/punch * h |
| C-51 | 1 | 22.2 | |
| C-52 | 2 | 14.2 | 40.7 |
| C-53 | 3 | 43.1 | 59.7 |
| C-54 | 4 | 28.9 | 44.6 |
| C-55 | 5 | 26.3 | 43.5 |
| C-56 | 6 | 30.1 | 56.2 |
| C-57 | 7 | 16.3 | 39.3 |
| C-58 | 8 | 26.9 | 48.4 |
| C-59 | 9 | 33.2 | 56.1 |
| C-60 | 10 | 25.4 | 45.4 |
| C-61 | 11 | 25.0 | 39.6 |
| C-62 | 12 | 36.2 | 49.3 |
| C-63 | 13 | 22.9 | 41.5 |
| C-64 | 14 | 16.1 | 32.9 |
| C-65 | 15 | 32.3 | 53.7 |
| C-66 | 16 | 17.0 | 25.5 |
| C-67 | 17 | 34.1 | 59.2 |
| C-68 | 18 | 29.6 | 49.6 |
| C-69 | 19 | 21.6 | 40.1 |
| C-70 | 20 | 36.6 | 58.0 |
| C-71 | 21 | 30.4 | 49.9 |
| C-72 | 22 | 30.8 | 38.3 |
| C-73 | 23 | 30.6 | 35.5 |
| C-74 | 24 | 30.6 | 42.1 |
| C-75 | 25 | 50.4 | 45.3 |
| C-76 | 26 | 29.5 | 53.9 |
| C-77 | 27 | 29.5 | 50.5 |
| C-78 | 28 | 20.5 | 28.2 |
| C-79 | 29 | 21.7 | 34.2 |
| C-80 | 30 | 31.7 | 42.8 |
| C-81 | 31 | 44.7 | 49.8 |
| C-82 | 32 | 44.7 | 48.4 |
| C-83 | 33 | 34.3 | 38.7 |
| C-84 | 34 | 29.1 | 36.3 |
| C-85 | 35 | 23.4 | 41.2 |
| C-86 | 36 | 23.7 | 49.0 |
| C-87 | 37 | 16.5 | 49.9 |
| C-88 | 38 | 19.9 | 46.5 |
| C-89 | 39 | 10.8 | 38.6 |
| C-90 | 40 | 16.1 | 36.1 |
| C-91 | 41 | 12.4 | 35.6 |
| C-92 | 42 | 20.6 | 47.1 |
| C-93 | 43 | 9.0 | 35.1 |
| C-94 | 44 | 10.6 | 27.8 |
| C-95 | 45 | 12.2 | 40.5 |
| C-96 | 46 | 21.0 | 53.0 |
| C-97 | 47 | 12.3 | 36.2 |
| C-98 | 48 | 20.3 | 57.4 |
| C-99 | 49 | 12.5 | 38.3 |
| C-100 | 50 | 14.1 | 39.7 |
| C-101 | 51 | 12.4 | 26.4 |
| | Median | 23.7 | 42.5 |

Although the invention has been illustrated and described with respect to one or more implementations, equivalent alterations and modifications will occur to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

The Abstract of the disclosure will allow the reader to quickly ascertain the nature of the technical disclosure. It is submitted with the understanding that it will not be used to interpret or limit the scope or meaning of the following claims.

What is claimed is:

1. A biomarker for the diagnosis of early kidney disease comprising: alpha-galactosidase, mutants, fragments, variants, derivatives or enzymatic activity thereof.

2. The biomarker of claim 1, wherein alpha-galactosidase activity is modulated in patients versus normal healthy controls.

3. The biomarker of claim 1, wherein detection of alpha-galactosidase activity, is correlated with different stages of kidney diseases.

4. The biomarker of claim 1, wherein detection of alpha-galactosidase activity differentiates between healthy individuals and individuals at stage one early chronic kidney disease.

5. A biomarker for the diagnosis of acute renal failure comprising: alpha-galactosidase, mutants, fragments, variants or derivatives thereof.

6. The biomarker of claim 5, wherein levels of alpha-galactosidase activity, is correlated with different stages of kidney diseases.

7. The biomarker of claim 5, wherein levels of alpha-galactosidase activity is diagnostic of acute renal failure.

8. A biomarker for measuring kidney function in vivo, comprising: alpha-galactosidase, mutants, fragments, variants, derivatives or enzymatic activity thereof.

9. The biomarker of claim 8, wherein normal alpha-galactosidase expression, function or activity is correlated with normal kidney function in vivo as compared to an abnormal or diseased kidney.

10. The biomarker of claim 8, wherein alpha-galactosidase expression, function or activity is diagnostic of kidney diseases or disorders, comprising: inflammation, infection, kidney transplantation, proteinuria, Fabrys disease, nephritis, congenital hydronephrosis, duplicated ureter, Horseshoe kidney, polycystic kidney disease, renal agenesis, renal dysplasia, unilateral small kidney, multicystic dysplastic kidney, diabetic nephropathy, glomerulonephritis, hydronephrosis, interstitial nephritis, nephrolithiasis, kidney tumors, Wilms tumor, renal cell carcinoma, Lupus nephritis, nephrotic syndrome, pyelonephritis, renal failure, acute renal failure or chronic kidney disease.

11. The biomarker of claim 8 wherein modulation of alpha-galactosidase in vivo is diagnostic of a disease or disorder.

12. A method of treating kidney diseases or disorders in vivo, comprising: administering to a patient in need thereof, a therapeutically effective amount of alpha-galactosidase.

13. The method of claim 12, wherein kidney diseases or disorders, comprising: inflammation, infection, kidney transplantation, proteinuria, Fabrys disease, nephritis, congenital hydronephrosis, duplicated ureter, Horseshoe kidney, polycystic kidney disease, renal agenesis, renal dysplasia, unilateral small kidney, multicystic dysplastic kidney, diabetic nephropathy, glomerulonephritis, hydronephrosis, interstitial nephritis, nephrolithiasis, kidney tumors, Wilms tumor, renal cell carcinoma, Lupus nephritis, nephrotic syndrome, pyelonephritis, renal failure, acute renal failure or chronic kidney disease.

14. A method of identifying biomarkers for diagnosis of early stage chronic kidney disease comprising: obtaining samples from patients;
measuring alpha-galactosidase activity;
correlating the alpha-galactosidase activity with each stage of kidney disease;
identify biomarkers expression, function and/or activity and correlated with a stage of kidney disease; and,
identifying biomarkers for diagnosis of early stage chronic kidney disease.

15. The method of claim 14, wherein the alpha galactosidase activity decreases between each stage with stage 1 having higher activity as compared to each progressive stage of chronic kidney disease.

16. A method of identifying biomarkers for diagnosis of acute renal failure and/or identification of individuals at risk of acute renal failure comprising:
obtaining samples from patients;
measuring alpha-galactosidase activity;
correlating the alpha-galactosidase activity with each stage of kidney disease;
identify biomarkers which are expressed or an activity of a molecule is detected and correlated with the stage of kidney disease; and,
identifying biomarkers for diagnosis of acute renal failure and/or identification of individuals at risk of acute renal failure.

17. A method of diagnosing and differentiating between patients at different stages of kidney disease comprising:
detecting alpha-galactosidase activity and/or a biomarker; and,
diagnosing and differentiating between patients at different stages of kidney disease.

18. The method of claim 17, wherein alpha-galactosidase activity is modulated during each stage of kidney disease.

19. The method of claim 17, wherein alpha-galactosidase activity decreases at late stages and acute renal failure.

20. A method of identifying agents for treatment or prevention of kidney diseases or disorders comprising:
contacting a biomarker indicative of kidney disease with an agent;
measuring activity, expression and/or function of the biomarker in the presence or absence of the agent;
correlating the biomarker activity with a normal control; and,
identifying an agent for the prevention or treatment of kidney diseases or disorders.

21. A method of identifying agents for treatment or prevention of diseases or disorders associated with alpha-galactosidase expression, function and/or activity, comprising:
contacting a sample with an agent;
measuring activity, expression and/or function of alpha-galactosidase in the presence or absence of the agent;
correlating the activity, expression and/or function of alpha-galactosidase with a normal control; and,
identifying an agent for the prevention or treatment of diseases or disorders associated with alpha-galactosidase expression and/or activity.

22. The method of claim 21, wherein the alpha-galactosidase expression, function and/or is optionally further correlated with one or more biomarkers.

23. The method of claim 21, wherein a sample comprises: a cell, tissue, fluid, peptide, polypeptide, oligonucleotide, polynucleotide, organic molecules, inorganic molecules or combinations thereof.
